(19) United States
(12) Patent Application Publication
(10) Pub. No.: US 2025/0014705 A1
(43) Pub. Date: Jan. 9, 2025

(54) MEDICAL DEVICE FOR CONTROLLED DISPENSING AND ADMINISTRATION OF A DRUG PRODUCT PRONE TO MISUSE AND ABUSE

(71) Applicant: NOVUS-MEDVICE INC., Powell, OH (US)

(72) Inventors: Praneeth Rao KAKULLAMARRI, Powell, OH (US); Rushil Reddy YELMA, Beaverton, OR (US); Praneeth MANDA, Elkridge, MD (US)

(73) Assignee: NOVUS-MEDVICE INC., Powell, OH (US)

(21) Appl. No.: 18/761,980

(22) Filed: Jul. 2, 2024

Related U.S. Application Data
(60) Provisional application No. 63/511,747, filed on Jul. 3, 2023.

(51) Int. Cl. G16H 20/13; A61M 5/00; G16H 10/60; G16H 40/67

(52) U.S. Cl. CPC G16H 20/13; A61M 5/00; G16H 10/60; G16H 40/67; A61M 2205/8262

(57) ABSTRACT

A programmable medical device is disclosed that includes at least one or more of the following: a housing having at least one compartment configured to store a drug, a drug delivery unit mounted within the housing, at least one biometric reader, a sensor configured to measure an angle at which the medical device is being held and a control circuit connected to the drug delivery unit, the at least one biometric reader, and the sensor. Systems that incorporate the medical device and methods for using the medical device are also disclosed.

### CROSS-REFERENCES TO RELATED APPLICATIONS

This application claims the benefit of U.S. Provisional Application 63/511,747, filed Jul. 3, 2023, which is incorporated by reference in its entirety.

### FIELD

The present disclosure relates to a programmable medical device for controlled, authorized dispensing and/or administration of a drug product prone to misuse and abuse and systems that include the medical device.

### BACKGROUND INFORMATION

Drug abuse is an ongoing worldwide crisis. According to the United Nations Office on Drugs and Crime World Drug Report for 2022, drug use accounted for about 5% of all substance related deaths worldwide in 2019. About 77% of all drug-related deaths were caused by opioid abuse. Indeed, opioids remain the most lethal group of drugs, accounting for two thirds of deaths related directly to drugs (mostly overdoses) per year. In North America alone, overdose deaths, driven by the use of fentanyls, reached unprecedented levels during the COVID-19 pandemic.

Opioids, which are a type of narcotic, are highly addictive pain medications that can lead to drug addiction. Drug addiction is a chronic disease characterized by drug seeking and use that is compulsive, or difficult to control, despite the harmful consequences that can occur with repeated use of the drug. The initial decision to take drugs is voluntary for most people, especially in circumstance where an individual is suffering from some form of chronic or temporary pain. Repeated drug use, however, has been known to lead to brain changes that can alter or challenge a user's self-control and interfere with their ability to resist intense urges to take a drug outside of its prescribed dosing regimen. These brain alterations can persistent after the user's drug treatment has ended and their supply has been emptied. Indeed, drug addiction is considered a “relapsing” disease, wherein users in recovery from drug use disorders are at increased risk for returning to drug use even after years of not taking the drug. Drug addiction can ultimately lead the user to seek unlicensed sources of the drug. The use of drugs from unlicensed sources is highly dangerous since there are no quality control measures in place to prevent the sale or distribution of counterfeit drugs which are often falsely or deceptively packaged or marketed as a substance approved by the Food and Drug Administration (FDA). There are also no quality control measures in place to prevent the sale or distribution of drugs laced with other chemicals, such as fentanyl.

Fentanyl abuse has reached unprecedented levels in North America. The increase in fentanyl abuse can be tied to its white powdery appearance and its odorless, tasteless properties, which allows it to easily be blended with other drugs, such as Xanax, Oxycodone, Ecstasy, Percocet and Adderall, to name a few.

Fentanyl is a synthetic opioid that is up to 50 times stronger than heroin and 100 times stronger than morphine, thus making it a highly dangerous opioid, especially when drug users are not aware of its presence. Even in small doses, fentanyl has been shown to be deadly.

Until recently, substance misuse problems and abuse disorders were viewed as personal, family, or social problems that were best managed at the individual and family levels. Programs for treating addiction were created with resources such as administration, regulation, and financing purposely placed outside public healthcare providers and systems. This meant that, with the exception of hospital-based detoxification, virtually all treatment was delivered by programs that were geographically, financially, culturally, and organizationally separate from mainstream healthcare. The policies of these programs focused treatment only on individuals with serious addiction, thereby providing few provisions for detecting or intervening clinically with far more prevalent cases of early-onset, mild, or moderate substance use disorders. Though the creation of these systems was a step toward addressing substance use problems, their policies created unintended and enduring impediments to the quality and range of care options for patients suffering from serious addition and patients suffering from early onset to moderate addiction. For example, within general healthcare, efforts to reduce the costs of hospital stays and surgical procedures led insurers to increase pharmacy benefits to stimulate discovery of new medications; whereas in the addiction field, treatment was already inexpensive and since there were far fewer physicians providing care, there were no pharmacy benefits. Consequently, there were very few medications to treat addictions.

Though there has been progress in developing programs for treating and deterring drug addiction and abuse, the overprescription of drugs prone to abuse and misuse is still a prevalent problem contributing to the drug abuse crisis seen worldwide. In the US alone, there were 240 million opioid prescriptions dispensed in 2015, nearly one for every adult in the general population. Physicians have also been known to overprescribe drugs, such as opioids and antibiotics, from following conventional prescription practices. For example, physicians are used to prescribing drugs in doses of multiples of 30 (30 days for a month supply of a once-a-day medication) when only about 5 to 20 drug doses would have been sufficient. These prescription practices lead to patients storing their leftover drug doses in places where they can be easily forgotten and found by other individuals who may sell, use, and/or seeking to abuse the drug. Another potential problem is that the patient may believe that they need to take all of the drug doses prescribed to them even when they are no longer suffering, thereby increasing their chance of developing drug addiction and abuse.

Adoption of smart dispensing devices is necessary to bridge the gaps underlying the conventional prescription drug dispensing practices contributing to drug addiction, abuse/misuse and overdose. Some of the many underlying factors contributing to prescription drug addiction and overdose are uncontrolled accessibility of all the doses prescribed to an user, unavailability of a medicine tracking or locating feature when needed in an emergency situation, unavailability of an antidote or symptom relieving drug tracking or locating feature when needed in an emergency situation, diversion of the prescription drugs, no rational or evidence based medication tool (e.g., a software, computer model or other device that can assist in determining prescriptions) for doctors or physicians to use in cases of prescribing addiction prone drugs, especially for pain which cannot be measured except on a visual analog scale, non-assistive drug dispensing, no tools to determine if drugs stored in the conventional bottles within the recommended storage conditions, no tool for detecting whether the bottles or containers in which the drugs are dispensed have been tampered, use of unused or expired drugs by family or friends and lack of enhanced patient prescription adherence.

To address the ongoing drug abuse and addiction crisis, various drug products with abuse deterrent properties and delivery devices have been produced to help prevent the onset of drug addiction and abuse, especially addiction and abuse associated with prescribed opioids.

WO 2017/174672 discloses auto-injectors possessing a re-useable drive unit comprising an electrically powered source of axial drive and a cassette unit comprising a syringe. The drive unit can have one or more inputs for receipt of data and/or commands relating to the operation thereof. These drug delivery devices are configured for administration of drug solutions and liquids.

CN 114566162 also discloses a drug delivery device configured for administering drug solutions and liquids. These devices have an intelligent anesthetic drug injection pump with a voice recognition function and an injection control method. The voice recognition module can be configured to send a control command that is associated with memristor resistance value information corresponding to the patient's personal information. The control command is sent to a motor drive control module that, upon verification of the patient, will dispense a first or second injection.

U.S. Pat. No. 11,213,624 discloses a drug delivery system that includes a drug delivery device having a reservoir, a delivery cannula having a proximal end in fluid communication with the reservoir and a distal end to be received within a patient, and one or more controllable elements. The drug delivery device is controlled, for the most part, based on one or more conditions and/or operational states of the drug delivery device determined through use of one or more onboard sensors.

CN 113257385 discloses a medicine injection system based on image and voice recognition, which comprises: a voice recognition module used for recognizing voice instructions, voice instructions that include a withdrawal instruction, a medicine injection instruction and an injection stopping instruction. An image acquisition module is used for acquiring an image of an injection site.

Though drug delivery devices have been produced to prevent the occurrence of drug abuse and addiction, these diseases/disorders are still prevalent worldwide. Additionally, many of the drug delivery devices have been designed for use by patients that do not suffer from mobility and dexterity issues. Many patients that are prescribed drugs belong to the geriatric population, (i.e., those 65 years and older). These individuals typically suffer from limited motor skills and could have trouble handling a medical device designed to administer their prescriptions. Additionally, some of these individuals typically suffer from dysphagia (difficulty swallowing food and liquids). The current medical devices configured to deliver oral dosage forms of prescription drugs on the market are not designed to address this issue.

Accordingly, to address the ongoing worldwide drug abuse and addiction crisis and to provide a more user-friendly drug delivery experience, a medical device for controlled administration of a drug product and systems designed to improve the medical device's controlled administration of the drug product are provided.

### SUMMARY

Disclosed herein is a programmable medical device for controlled, authorized administration of a drug that comprises: (a) a housing that includes at least one compartment configured to store a drug, (b) a drug delivery unit mounted within the housing that is configured to store a therapeutic dose of the drug from the at least one compartment and dispense the therapeutic dose to an authorized user, (c) at least one biometric reader configured to capture a physical feature of the authorized user, (d) a sensor configured to detect an angle at which the medical device is being held by the authorized user, and (e) a control circuit configured to (i) communicate with the sensor and verify that the angle detected by the sensor is within a preset range of angles and, when the angle is verified as falling within the preset range of angles, (ii) provide power to the at least one biometric reader and the drug delivery unit, wherein once the at least one biometric reader captures the physical feature of the authorized user and the control circuit verifies that the physical feature belongs to the authorized user, the control circuit instructs the drug delivery unit to dispense the therapeutic dose of the drug from the drug delivery unit to the authorized user.

Disclosed herein is also a system for controlled, authorized administration of a drug. The system comprising: (a) a medical device having a compartment configured to store one or more therapeutic doses of a drug, a drug delivery unit configured to dispense at least one therapeutic dose of the drug to an authorized user, at least one biometric reader configured to capture a physical feature of the authorized user, a sensor configured to measure an angle at which the medical device is being held, and a control circuit configured to (i) verify that the angle detected by the sensor is within a preset range of angles and, when the angle at which the medical device is being held is verified as falling with the preset range of angles, (ii) provide power to the at least one biometric reader and the drug delivery unit, wherein once the at least one biometric reader captures the physical feature of the authorized user and the control circuit verifies that the physical feature belongs to the authorized user, the control circuit instructs the drug delivery unit to dispense the therapeutic dose of the drug from the drug delivery unit to the authorized user; (b) a device configured to measure the authorized user's vitals and communicate with the medical device, and (c) a remote device configured to (i) wirelessly communicate with the control circuit of the medical device and (ii) execute an application program interface that allows the authorized user and a third-party to monitor a status of the medical device and adjust at least one of settings and control parameters of the medical device.

Disclosed herein is also a method for dispensing a drug through a medical device that includes (a) a housing having at least one compartment configured to store a drug, (b) a drug delivery unit mounted within the housing, (c) at least one biometric reader, (d) a sensor configured to measure an angle at which the medical device is being held and (e) a control circuit connected to the drug delivery unit, the at least one biometric reader, and the sensor, the method comprising: (i) detecting, by the sensor, an angle at which the medical device is being held by the authorized user, (ii) verifying, by the control circuit, that the angle detected by the sensor is within a preset range of acceptable angles, (iii) powering, by the control circuit, the at least one biometric and the drug delivery unit, (iv) verifying, by the control circuit, an identity of the authorized user based on a physical feature captured by the at least one biomarker reader, and (iv) instructing, by the control circuit, the drug delivery unit to dispense the therapeutic dose of the drug to the authorized user when both the identity of the authorized user is verified and the angle detected by the sensor is held is verified as falling with the preset range of acceptable angles.

### BRIEF DESCRIPTION OF THE DRAWINGS

FIG. 1 shows exemplary embodiments of the components of the programmable medical device.

FIG. 2 shows a first exemplary embodiment of the programmable medical device.

FIG. 3A depicts a flowchart of an exemplary process of how the programmable medical device controls the administration of the drug contained in the drug storage compartment.

FIG. 3B depicts a second exemplary embodiment of the programmable medical device containing a biometric lip reader on a mouthpiece.

FIG. 4 shows a third exemplary embodiment of the programmable medical device.

FIG. 5 shows a fourth exemplary embodiment of the programmable medical device.

FIG. 6 shows a fifth exemplary embodiment of the programmable medical device.

FIG. 7 shows an exemplary embodiment of a system including a programmable medical device and a remote device.

FIG. 8 depicts a flowchart of an exemplary process of how the programmable medical device controls the administration of the drug contained in the drug storage compartment and notifies the authorized user and a caregiver of an unauthorized attempt to access the drug in the drug storage compartment.

FIG. 9 depicts a flowchart of an exemplary process of how the programmable medical device indicates that the drug contained in the drug storage compartment is not ready for administration and distinguishes whether the authorized user or an unauthorized user attempted to access the drug before the drug was ready for administration.

Further areas of applicability of the present disclosure will become apparent from the detailed description provided hereinafter. It should be understood that the detailed description of exemplary embodiments is intended for illustration purposes only and is, therefore, not intended to necessarily limit the scope of the disclosure.

### DETAILED DESCRIPTION

The exemplary embodiments described in the present disclosure pertain to a drug dispensing medical device which can be programmed to dispense drugs to an authorized user at predetermined intervals by doctor/physician, enable the authorized user to trace or locate the drugs stored within the device in emergency situations, store a life-saving antidote or adverse effects reversal drug within the device so the authorized user and/or a caregiver can trace or locate the life-saving antidote or adverse effects reversal drug in emergency situations (thereby saving time and, possibly, the life of the authorized user or another individual before medical intervention arrives), dispense the drugs directly into the mouth of the authorized user while simultaneously/concurrently identifying the user (thereby deterring the authorized user from sharing the drugs with friends, family or other individuals), read the vitals of the authorized user and control the dispensing of the drug from the gathered vitals data (thereby providing a tool for rationale prescribing of medicines like opioids for pain which cannot be physically measured and where the prescriber has to rely on user/patient inputs), assist users with limited dexterity (e.g., geriatric users), sense whether the conditions of the stored drug products or device are outside the recommended storage conditions of the drug products, notify the authorized user when the drug products or device is stored at conditions outsides the recommended storage conditions of the drug products stored in the device, determine the duration of the time that has passed since the drug product was last administered, determine the duration of time the drug products or device has been stored in conditions outside the recommended storage conditions, determine whether the device has been tampered with or determine whether an attempt of tampering with the device has occurred, determine the amount of unused drug product or expired drug product in the device, destroy any amount of unused drug product or expired drug product in the device to deter abuse or misuse of the drug product by friends, family or other individuals and/or enhance user prescription adherence.

The present disclosure also describes exemplary programmable medical devices, exemplary systems that employ these medical devices to better control and monitor the administration of the drugs stored in the medical devices, and exemplary methods for dispensing a drug through the programmable medical devices. A detailed disclosure of the devices, systems and methods are provided herein below.

### Medical Devices

According to exemplary embodiments of the present disclosure, the programmable medical devices include one or more of the following components: a housing that includes at least one compartment configured to store a drug, a drug delivery unit mounted within the housing that is configured to dispense a therapeutic dose of the drug from the at least one compartment to an authorized user, at least one biometric reader configured to capture a physical feature of the authorized user, a sensor configured to detect an angle at which the medical device is being held by the authorized user, and a control circuit configured to (i) communicate with the sensor and verify that the angle detected by the sensor is within a preset range of angles and, when the angle at which the medical device is being held is verified, (ii) provide power to the at least one biometric reader and the drug delivery unit, wherein once the at least one biometric reader captures the physical feature of the authorized user and the control circuit verifies that the physical feature belongs to the authorized user, the control circuit instructs the drug delivery unit to dispense the therapeutic dose of the drug from the drug delivery unit to the authorized user.

As used herein, the term “therapeutically effective dose” refers to an amount of a drug product or medication that is sufficient to cure or at least partially alleviate symptoms associated with a disease and/or pain in a user suffering from the disease and/or pain.

The drug that can be stored in at least one compartment of the housing and administered to the authorized user can be any drug or drug product that is prone to abuse and or misuse either when taken alone or taken with other drugs that can enhance the effect of the abuse or misuse of the drug. These drugs or drug products can include, but are not limited to any one or more of the following: an opioid (i.e., codeine, morphine, oxycodone, opioid hydrocodone), an antihistamine, an antidepressant, a stimulant (i.e., amphetamines, methylphenidates) a barbiturate, a benzodiazepine, a sleep medication (i.e., zolpidem, eszopiclone, zaleplon), acetaminophen, dextromethorphan, cannabinoids, and/or pseudoephedrine. The foregoing list provides exemplary drug and drug product candidates that can be dispensed by the medical device described herein. It should be understood that the list can be expanded to include additional drugs as desired, for example, highly potent drugs such as Fentanyl and Sufentanyl and/or other drugs acting on the central nervous system (CNS). According to exemplary embodiments described herein the drug can be in a-solid form, such as a pill, tablet, caplet, capsule, chewable gel or any other suitable solid composition as desired. In other examples, the drug or drug product can be in a semi-solid form, such as medications applied to the skin, nasal mucosa, cornea, buccal tissue, ear, or other soft tissue areas of the body as desired. According to yet another exemplary embodiment of the present disclosure, the drug can be in a liquid form.

As used herein, the term “authorized user” refers to an individual that has had at least one physical feature logged onto any of the medical devices described herein and is recognized by any of the medical devices as being the authorized recipient of the drug stored in the medical device. This individual can be suffering from symptoms associated with a disease and/or pain, been legally prescribed a drug product by a licensed medical provider for treatment in alleviating said symptoms and uses any of the medical devices described herein to control the administration of the prescribed drug.

As used herein, the term “unauthorized user” refers to an individual that has not had at least one physical feature logged onto any of the medical devices described herein and is not recognized by any of the medical devices as being the authorized recipient of the drug stored in the medical device.

The medical devices described herein can be configured to capture and log at least one physical feature of a user through one or more biometric technologies using the user's fingerprint, the user's facial structure, the user's periocular region (area surrounding the eyeballs), the user's eyes (retina), the user's lip pattern (cheiloscopy), the user's voice, and/or any other physical features suitable for determining the identity of a user as desired.

The medical devices described herein can include a sensor configured to sense an angle of a slope, elevation, or depression at which the medical device is positioned or held by a user. The administration of a drug can be dependent on the detected angle, i.e., the drug is administered once the medical device is held at a specified angle or within a predetermined range of acceptable angles. The medical devices can be programmed to, once an acceptable angle has been detected, capture a physical feature of the user, verify an identity of the user based on the captured feature, and dispense the drug to the authorized user via the drug delivery unit based on the user's verified identity. According to an exemplary embodiment, the detection and verification of the angle and the capture of a user's physical feature and verification of the user's identity can be performed in parallel or in sequence.

In exemplary embodiments, the control circuit can instruct the drug dispensing unit to dispense or administer a drug to a user once the medical device is held at an acceptable angle. In one example, the acceptable angle can be any angle within the range of 0° to 90° or 0° to 45°. Once the control circuit verifies that the detected angle of the medical device is within the acceptable range, the control circuit can activate or control the at least one biometric reader to capture a physical feature of the user. For example, the at least one biometric reader can include a sensor configured to capture a facial image of the user. The sensor can be mounted on the medical device such that the angle at which the medical device is held, impacts the ability of the biometric reader to capture an image suitable for the control circuit to perform a facial recognition process. The sensor that can be configured to detect the angle at which the devices are positioned can be located within the housing of the medical devices. According to exemplary embodiments, the sensor can include one or more of a rotary position sensor, an angular sensor, or any other suitable sensor type as desired.

According to exemplary embodiments described herein the medical device can be programmed to not activate or deactivate, at any time, the dispensing of the drug directly into the drug delivery unit, the identifying of a physical feature of the authorized user, and/or the administration of the drug from the drug delivery unit, if the sensor (configured to sense an angle of a slope, elevation, or depression at which the medical device is positioned or held by the authorized user) detects that the angle at which the medical device is being held is outside of the predetermined range of acceptable angles. This feature of the medical device aids in ensuring that the drug is being dispensed orally to an authorized user through a mouthpiece and also serves as a deterrence mechanism to prevent the authorized user from sharing the drug with friends, family or other individuals since the medical device must (i) remain being held at an acceptable angle and (ii) be held close enough to the authorized user for the biometric reader to successfully capture a physical feature of the authorized user for the drug to be administered.

In exemplary embodiments, the medical device can include at least two biometric readers. One biometric reader can be located at the distal end of the housing (the end farthest from the drug delivery unit) and the other can be positioned near the drug delivery unit, and preferably on a mouthpiece of the medical device. The biometric reader located at the distal end of the housing can be configured to capture a facial feature of the authorized user. The biometric reader located on the mouthpiece of the medical device can be programmed to capture a lip print of the authorized user. As already discussed, the control circuit can be programmed to first detect the angle at which the medical device is being held and determine whether the angle falls within a predetermined range of acceptable angles and, once the control circuit determines that the medical device is being held at an acceptable angle, it can activate (e.g., simultaneously power) the at least two biometric readers and the drug delivery unit. Once the physical features of the authorized user have been verified and the drug enters into the drug delivery unit, the drug delivery unit can pass the therapeutic dose of the drug through the mouthpiece and administer the drug to the authorized user. In exemplary embodiments, the control circuit can be configured to halt the administration of the drug, the verification of the authorized user and/or the transfer of the drug into the drug delivery unit if the control circuit determines that the medical device is no longer being held at an acceptable angle at which a biometric reading of the user can be captured.

The exemplary medical device described herein can be configured to continuously monitor the position of the medical device as detected by the sensor. For example, the control circuit can be configured to continuously monitor or, at specified intervals, periodically monitor an output of the sensor configured to detect an angle of slope, elevation, or depression at which the medical device is being held by a user. The control circuit can compare the output of the sensor to a predetermined range of acceptable angles and activate or deactivate one or more light emitting diodes (LEDs) and/or auditory cues which notify the user that the medical device is primed or not primed for drug administration.

In exemplary embodiments, the medical device can include at least one driving unit within the housing, wherein the at least one driving unit is configured to drive (e.g., move) one or more components of the drug delivery unit to administer the drug to the authorized user. In addition, the at least one driving unit can be configured to prevent a drug from entering the drug delivery unit from at least one storage compartment in the housing when the drug is not ready or primed for administration and when an unauthorized user attempts to use the medical device. For example, the at least one compartment can be configured to include a tray having plural chambers, wherein each chamber is configured to store a therapeutic dose of the drug. The driving unit can be configured to rotate the tray during a dispensing operation such that one of the plural chambers containing a dose of the drug is aligned with an opening in the at least one compartment. According to an exemplary embodiment, the driving unit can include a small/mini electric motor such as a stepper motor, a brushless DC motor, a brushed DC motor, or any other suitable motor as desired.

In exemplary embodiments, the medical device can include at least one circuit board having a control circuit to control and monitor an operation of the medical device, wherein one or more components for performing control, monitoring, and/or processing operations is mounted to the at least one circuit board. The control circuit can be configured with program code to control the dispensing operation of the medical device by activating the at least one driving unit. For example, the control circuit can be configured to activate the motor to rotate the tray and align one of the plural chambers with an opening of the at least one compartment. According to an exemplary embodiment, the activation of the motor can be dependent on the control circuit verifying an identify of the user. The user can be verified by comparing a physical attribute obtained from the user through at least one biometric reader with one or more reference attributes stored in memory. According to another exemplary embodiment, the activation of the motor can be dependent on the control circuit determining whether the medical device is held at a specified position for dispensing the therapeutic dose of the drug through a mouthpiece. For example, the control circuit can be configured to receive a position measurement from at least one position sensor of the medical device and determine whether the position of the medical device is at a specified angle or within an acceptable range of angles. According to yet another exemplary embodiment, the dispensing operation which includes activation of the driving device can be dependent on whether the control circuit verifies that a pre-defined time interval is met or has elapsed since the drug was last administered or dispensed to the user. It should be readily apparent that the control circuit can execute the dispensing operation using one or a combination of the aforementioned techniques to activate the driving device or motor.

The pre-defined time interval can be any suitable time frame determined based on the prescription issued by the medical professional, the type of drug being administered, physical condition of user based on vital readings, or any other data or information as desired. According to exemplary embodiments, the pre-defined interval can fall anywhere between 0 to 24 hours after the drug was last administered, such as at least 4 hours after the drug was last administered, at least 6 hours after the drug was last administered, at least 12 hours after the drug was last administered or at least 24 hours after the drug was last administered. In exemplary embodiments, the pre-defined time interval is based on the half-life of the drug stored in the at least one drug storage compartment. Those skilled in the art would readily be able to determine the half-life of a drug and program the medical device to administer the drug in pre-defined time intervals based on the drug's half-life.

In exemplary embodiments, the medical device can be programmed to notify the authorized user that a pre-defined time interval has passed and that the drug is ready for administration. The medical device can be programmed to perform an auditory and/or visual cue to notify the authorized user that the drug is ready for administration.

According to an exemplary embodiment, the control circuit can be configured to deactivate the driving unit and disable the dispensing operation to prevent a drug from entering a mouthpiece under one or more conditions when the drug is not ready for administration and/or when an unauthorized user attempts to use the medical device. The control circuit can include one or more processing devices, such as a central processing unit, a microprocessor, microcomputer, programmable logic unit or any other suitable processing device that can be configured and/or encoded with software or program code as desired.

The circuit board(s) that can be include in the medical devices disclosed herein can be a printed circuit board (PCB) having any number of layers and materials suitable for supporting and providing electrical connection(s) between the components mounted thereon and between the components mounted on thereon and any suitable external devices as desired.

In exemplary embodiments, the medical device can include at least one battery configured to supply the medical device with power. The at least one battery can be mounted within the housing of the medical device. In exemplary embodiments, the at least one battery can be configured to supply a circuit board, a driving unit, a biometric reader, at least one sensor and/or at least one visual and/or auditory signaling component (e.g., a LED or a speaker) with operating power. The battery can include any suitable type of power source as desired including but not limited to alkaline batteries, nickel metal hydride batteries, and lithium-ion batteries.

In exemplary embodiments, the medical device can include a charging dock configured to mechanically engage with the housing and charge at least one battery mounted within the medical device. In exemplary embodiments, the charging dock can include connectors for providing an electrical connection to the at least one battery of the medical device. For example, the connectors can include one or more electrodes for connecting the battery to the control circuit and/or a power source. The charging dock can also include a control circuit configured to control and/or monitor the charge level of the medical device and the charging operation of the at least one battery mounted within the medical device. The control circuit can be separate from or include the control circuit that controls the dispensing operation of the medical device. The charging dock can include a charging port configured to communicate with and charge at least one battery mounted within the medical device and/or at least one battery located within the charging dock. The medical device can also include a charging port to provide electrical power for charging the battery at a maximum of 5V DC and between 500 mA to 3A. The housing of the medical device can include one or more types of charging ports, such as, Universal Serial Bus-A (USB-A), Universal Serial Bus-B (USB-B), Mini-USB, Micro-USB, USB-C, USB-3, or any other suitable charging port as desired.

The medical device of the present disclosure can include a locking device configured to prevent an authorized user and an unauthorized user from accessing a drug stored in at least one compartment of the medical device. According to an exemplary embodiment, the locking device can be disposed on a face or outer surface of the housing. For example, the locking device can be disposed on or in close proximity to at least one compartment of the medical device that is storing a drug. According to an exemplary embodiment, the locking device can be configured to allow access to the at least one compartment storing a drug when in communication (e.g., contact or contactless) with an authorized key. For example, the authorized key can be a physical key or an electronic key. The electronic key can be a smart card, a security token, a numerical combination for an electronic keypad and/or a RFID key fob, or any other suitable electronic or smart locking device as desired. The physical key can be a key cut to engage with and open a mechanical lock.

According to exemplary embodiments of the present disclosure, the one or more compartments of the programmable medical device can include plural storage units. One or more first storage units being configured to store the drug to be dispensed and one or more second storage units configured to store a life-saving product that counteracts potentially negative physical effects of the drug stored in the first storage unit. The second storage unit can be disposed in the housing of the medical device, in a charging dock configured to charge at least one battery in the medical device and/or in a compartment configured to be detachably connected to or removed from the medical device.

As used herein, the term “life-saving drug product” refers to a drug product that can reverse the symptoms associated with a drug overdose and/or symptoms associated with a disease state (e.g., diabetes). The life-saving drug product can be an opioid antagonist, insulin or an insulin analog, or any other suitable drug or product for reversing or counteracting negative physical effects of the drug to be or being administered by the device. According to an exemplary embodiment, the life-saving drug product can include at least one antidote, such as activated charcoal, acetylcysteine, naloxone, naltrexone, epinephrine, sodium bicarbonate, atropine, flumazenil and various vitamins, or any other suitable medicine or remedy for counteracting the negative physical effects of the drug to be or being administered by the device.

In exemplary embodiments, the programmable medical device includes at least one transceiver disposed within the housing and configured for wireless communication with a global positioning system (GPS). The control circuit can be configured to determine a location of the medical device based on geolocation data received from a GPS. The at least one transceiver can also be configured for wireless communication with a remote device of the user or other authorized third party, such as a licensed medical practitioner, a medical facility employee, a legal guardian and/or an individual that frequently communicates with the user (e.g., a friend or relative). According to an exemplary embodiment of the present disclosure, the at least one transceiver and the control circuit can be disposed in one of the plural compartments that is detachably connected to the medical device.

As used herein, the term “licensed medical practitioner” refers to an individual that possess an active medical license and/or an active pharmacy license and is aware of the authorized user's medical history.

In exemplary embodiments, the medical device includes a transceiver disposed within a second storage unit containing at least one life-saving drug product, the transceiver being configured for wireless communication with a global positioning system (GPS). An advantage of these embodiments is that the authorized user can better locate the life-saving drug product in emergency situations. The transceiver enables the authorized user or a caregiver to quickly and efficiently locate the medical device in critical situations, where the authorized user needs medical attention. The transceiver can include a beacon which broadcasts or transmits the location (e.g., GPS coordinates) of the medical device to a remote device of a caregiver or third party, thereby allowing them to more readily locate the storage unit and administer the life-saving drug product or provide appropriate emergency medical care. In exemplary embodiments, the medical device via the control circuit can be configured to output an auditory and/or visual cue to further help the authorized user and/or caregiver locate it.

In exemplary embodiments, the medical device can include at least one compartment configured to store a drug, the at least one compartment being a tablet hopper configured to store plural units of the drug, the plural units containing a therapeutic dose of the drug. The medical device can also include a door that is configured to be driven by a driving unit to allow one or more units of the drug to be dispensed from the at least one compartment when the driving unit is actuated by a control circuit and when at least one biometric of the authorized user has been verified, when the medical device is held within an acceptable range of angles and/or when a pre-defined time interval is met after the drug was last administered.

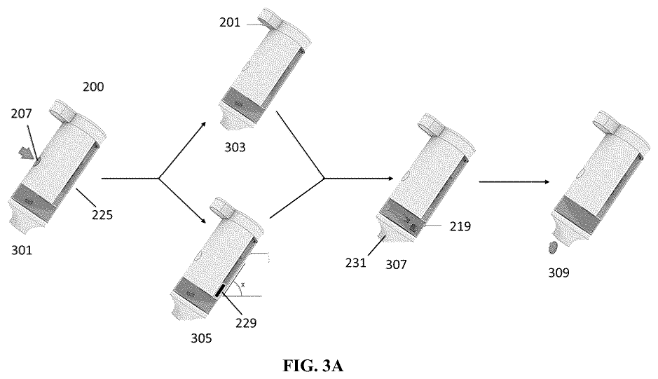

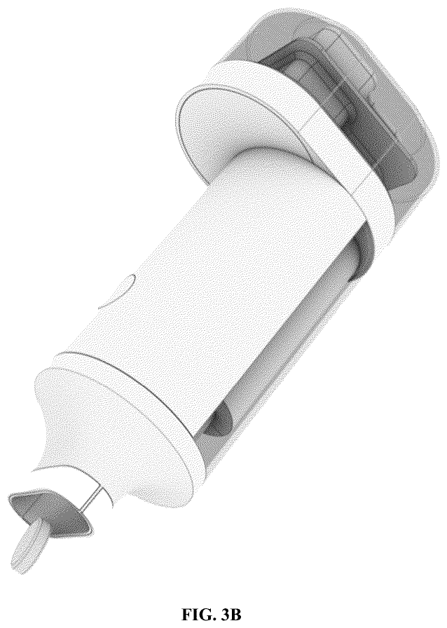

In exemplary embodiments, the medical device can include a mouthpiece configured to fit in the authorized user's mouth and dispense the therapeutic dose of the drug directly into the authorized user's mouth. The mouthpiece can be configured to include a biometric reader designed to capture the lip pattern of a user (see FIG. 3B). In these embodiments, the medical device via the control circuit can be configured to verify the identity of the user by comparing the captured lip pattern to a stored lip pattern of the authorized user.

In exemplary embodiments, the medical device can include at least one dosage splitting unit configured to split a therapeutic dose of a drug stored in the housing of the medical device into smaller dosages. According to an exemplary embodiment, the dosage splitting unit can be located between the at least one compartment storing the drug and the drug dispensing unit. The dosage splitting unit can include a blade configured to cut the drug into smaller doses or any other suitable device for reducing the size or dose of the drug to be or being administered by the device. According to an exemplary embodiment the dosage splitting unit can be manually activated and/or operated through user interaction or can be operated automatically via the driving unit. For example, the dosage splitting unit can be connected to the least one driving device (e.g., motor) or include a separate driving device that controlled and/or activated by the control circuit.

Some drugs available in the market and/or prescribed by physicians/doctors may require that they be split in halves or multiple pieces before ingesting. In general, tablets are split in halves either by hand or with tablet splitters available in pharmacies or online. An advantage of including a dosage splitting unit in the medical device is that the authorized user no longer needs to split the drug into smaller pieces by hand and/or with a tablet splitter. This feature thus allows for a more user-friendly drug delivery experience. Another advantage of including a dosage splitting unit in the medical device is that it can allow for an easier administration of the drug to authorized users that suffer from impaired mobility and/or dysphagia (e.g., geriatric users) since the doses of the drug are smaller.

In exemplary embodiments, the medical device can be a mobile or portable device. For example, the medical device can be of a suitable size, dimensions, and shape to be carried and/or held in a user's hand, stored in backpack, purse, or other personal storage item.

In exemplary embodiments, the programmable medical device includes a vital detection unit configured to obtain vital measurements from the authorized user. For example, the vital detection unit can include a wearable and/or re-usable biosensor that is to be attached to or placed in close proximity to a user's body or torso. According to an exemplary embodiment, the vital detection unit can include a photodiode and/or an accelerometer for detecting one or more of electrocardiography, heart rate, heart rate variability, heart rate respiratory, body temperature, physical activity, posture, and any other suitable or useful biometric as desired. In exemplary embodiments, the vital detection unit can be configured to communicate with and/or provide vital measurements to the control circuit of the medical device.

The control circuit and/or vital detection unit can also be configured to transmit the vital measurements of the authorized user to a licensed medical practitioner, a caregiver and/or an individual that communicates with the authorized user. In exemplary embodiments, the control circuit is programmed to receive vitals information of the authorized user from the vital detection unit and compare the vitals information received from the vital detection unit to one or more vital values stored in memory to determine whether the authorized user is in a condition for receiving the drug stored in the medical device. Transmittal of the authorized user's vital measurements to a licensed medical practitioner can allow for optimal administration of the drug since the licensed medical practitioner can more readily determine if the authorized user's vitals are stable before administration of the drug. Determining whether the vitals of the authorized user are stable before administration of the drug is advantageous since it lowers the likelihood of causing an adverse drug reaction from occurring from the administration of the drug to the authorized user when at least one vital is unstable or reaching critical levels.

The control circuit and/or vital detection unit can also be configured to notify the authorized user, a licensed medical practitioner, a caregiver and/or an individual that communicates with the authorized user that the authorized user's vitals are in a critical state. In exemplary embodiments, the vital detection unit and/or control circuit can activate an auditory and/or visual cue programmed into a storage unit of the medical device that contains a life-saving drug product so that the authorized user, a licensed medical practitioner, a caregiver and/or an individual that communicates with the authorized user can more readily locate the life-saving drug product and administer it to the authorized user. In exemplary embodiments, the medical device can be programmed to allow administration of the drug stored in the at least one compartment, even if the medical device and/or vital detection unit determines that the authorized user's vitals are in a critical state.

In exemplary embodiments, the medical device can include a sterilization unit configured to sterilize the drug delivery unit and/or a mouthpiece before and/or after dispensing a therapeutic dose of a drug to the authorized user. For example, the sterilization unit can include one or more sterilization products including napkins, towelettes, liquid or gel packets, spray, or any other suitable product for applying a sterilizing solution to the device mouthpiece and/or housing. The sterilization unit can include a light source capable of damaging and/or destroying bacteria and/or viruses (e.g., a UV light source). The sterilization unit can be located in a charging dock configured to charge the medical device, in a detachable unit configured to be reversibly removed from the medical device and cover the drug dispensing unit and/or mouthpiece, and/or located within the housing of the medical device.

In exemplary embodiments, the medical device can include at least one tamper detection sensor that is configured to detect an attempt at unauthorized access to the at least one compartment storing the drug, an attempt at unauthorized access of the drug delivery unit, and/or if the medical device or drug compartment has been physically compromised. An advantage of including this feature in the medical device is that it can allow a licensed medical practitioner/physician to better detect when drug abuse and addiction is occurring. With this information, the licensed medical practitioner/physician can more readily modify the drug prescription to the authorized user to better prevent further development of drug abuse and addiction.

The tamper detection sensor can be in the form of a polyvinylidene fluoride film (PVDF) disposed on a surface of the housing, a capacitive sensor, pressure sensor configured to detect whether a pressure change has occurred in the at least one compartment storing the drug, a humidity sensor configured to detect whether a humidity change has occurred in the at least one compartment storing the drug, or temperature sensor disposed within or in close proximity to the at least one compartment that is configured to detect whether a temperature change has occurred in or around the at least one compartment storing the drug, or any other suitable sensor as desired.

The at least one tamper detection sensor can be configured to detect at least one or more of the following tampering methods, which can compromise the physical integrity of the medical device and/or the drug compartment of the medical device: cutting of the housing of the medical device, breaking or crushing of the housing of the medical device, drilling a hole in the housing of the medical device, heating of the housing of the medical device, prying open the housing of the medical device and attempting unauthorized engagement of the dispensing mechanism of the medical device. The tamper detection sensor can be electrically connected to the processor provided on the circuit board for communicating readings based on the sensor configuration. The circuit board can include one or more memory devices for storing threshold tamper detection values. The processor can be configured to compare tamper detection readings received from the sensor to one or more tamper detection values stored in memory and determine whether a tampering event has occurred. For example, if the tamper detection sensor includes a pressure sensor, the one or more compartments storing the therapeutic drug can be pressurized to a pre-determined pressure value. In this case, the tamper detection threshold can be a pressure value which, if greater/lower than a tamper detection reading, indicates that a tampering event has occurred or that the physical integrity of the one or more compartments is comprised. According to another example, if the tamper detection sensor is a temperature sensor, the one or more compartments can be configured to store a gas that maintains a certain temperature. In this case, the tamper detection threshold can be a temperature value which, if less than a tamper detection reading, indicates that a tampering event has occurred or that the physical integrity of the one or more compartments is comprised. According to yet another example, if the tamper detection sensor is a capacitive sensor or PVDF film, the tamper detection threshold can be a sensor value which, if greater than a tamper detection reading received by the processor, indicates that a tampering event has occurred or that the physical integrity of the one or more compartments is comprised.

In exemplary embodiments, the medical device can include at least one compartment configured to store a drug, the at least one compartment including an inner shell and an outer shell, wherein the inner shell is a pressurized chamber, and the outer shell is at least part of the outer surface of the housing. The inner shell can be configured to detect pressure changes occurring in the housing.

In exemplary embodiments, the medical device can include a destruction unit located within the housing of the medical device, the destruction unit being configured to mechanically and/or chemically destroy an amount of drug remaining in the at least one compartment once activated by the authorized user and/or once a tampering attempt has been detected.

In exemplary embodiments, the destruction unit can be configured to crush and/or mill any amount of drug remaining in the at least one compartment. The medical device can further be configured to disable its drug administration functions once the drug stored in the at least one compartment has been crushed and/or milled.

In exemplary embodiments, the destruction unit can be further configured to release a chemical substance into the at least one compartment after the drug has been crushed and/or milled. The chemical substance can react with the crushed/milled drug product and create a solution that cannot be administered via intravenously and/or nasally due to either high viscosity and/or increased irritation to the user. Possible chemical substances include, but are not limited to, polyethyleoxide, hydroxy propyl cellulose, gel forming materials, silicon dioxide, sodium lauryl sulfates, acrylates and/or pH modifiers.

An advantage of including the destruction unit feature in the medical device is that it can better deter the authorized user from giving expired and/or unused amounts of the drug to friends, family and/or other individuals.

In exemplary embodiments, the medical device can include an activation unit configured to power on the medical device and activate the control circuit in the medical device.

In exemplary embodiments, the medical device includes one or more laser emitting diodes (LEDs) configured to display a status of the medical device. Activation of the one or more LEDs can be controlled by signals generated by the control circuit during the execution of one or more operations determined by program code.

In exemplary embodiments, the medical device can include one or more storage condition monitoring sensors configured to detect the environmental conditions in any storage compartment of the medical device. In exemplary embodiments, the storage condition monitoring sensors include a temperature sensor configured to detect the temperature of any storage compartment residing within the medical device. In other exemplary embodiments, the storage condition monitoring sensors include a humidity sensor configured to detect the humidity or moisture levels of any storage compartment residing within the medical device. The storage condition monitoring sensors can be located within the storage compartment they are monitoring and/or can be separate from and adjacent (above, below, beside) to the storage compartment they are monitoring.

In exemplary embodiments, the one or more storage condition monitoring sensors are configured to communicate with control circuit and, when the control circuit determines that the storage conditions in a storage compartment are outside a pre-set range of temperatures and/or humidity levels, the control circuit activates an audio and/or a visual cue that alerts the user to the deleterious conditions in the storage compartment. The range of temperatures and/or humidity levels that can be pre-set to detect the presence of deleterious conditions in any storage compartment in the medical device can be readily determined by those of ordinary skill in the art by looking at the drug label and/or safety data sheet of the drug product stored in the storage compartment.

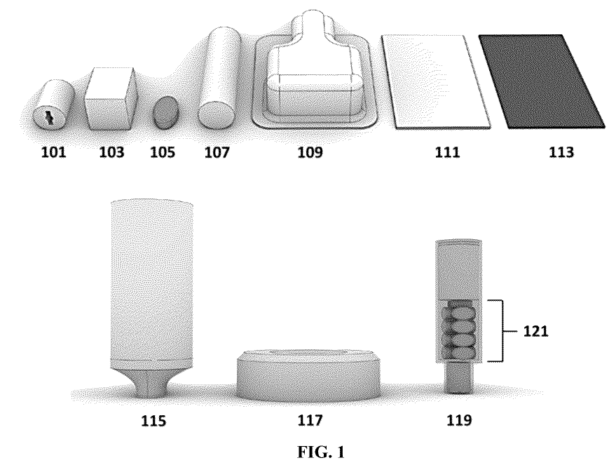

FIG. 1 depicts exemplary embodiments of the various components and features of a medical device in accordance with an exemplary embodiment. The components depicted are a lock device 101, a driving unit 103, a therapeutic dose of a drug 105, a battery 107, a storage unit configured to store a life-saving drug product 109, an optional testing strip configured to detect the presence of an impurity in a drug substance 111, a circuit board 113, a housing unit 115, a charging dock 117 and a drug compartment 121 connected to a drug dispensing unit 119.

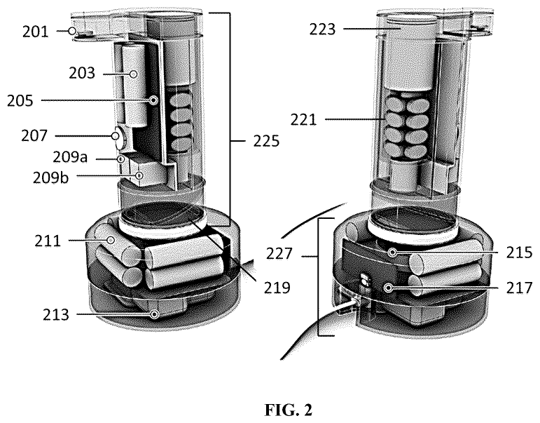

FIG. 2 depicts an exemplary embodiment of how the various components of FIG. 1 can be arranged or integrated into the medical device 200. As shown in FIG. 2, the medical device 200 can include a biometric reader 201 configured to capture a facial image of the authorized user and is located at a distal end of the housing 225. Batteries 203 are located within the housing 225 of the medical device 200 and are in communication with driving units 209a, 209b and circuit board 205. The housing 225 further contains an activation unit 207 configured to initiate the biometric reader 201 to capture a physical feature of the user and the medical device 200 to detect whether the drug stored in the drug storage compartment 221, located within the housing 225, is ready for administration. A drug dispensing unit 219 is located at the proximal end of the housing 225. The housing 225 also includes a locking device 223 configured to prevent unauthorized access of the drug in the drug storage compartment 221. The medical device 200 also includes a charging dock 227 that is configured to interact with the proximal end of the housing 225 and charge the batteries 203 located within the housing 225. The charging dock 227 contains batteries 211, a sterilization unit 215 configured to sterilize the drug dispensing unit 219 and a storage unit 213 configured to store a life-saving drug product. The charging dock 227 also contains a circuit board 217 that is configured to charge the batteries 203 located within the housing 225 when connected to an outside power source.

FIG. 3A depicts an exemplary process of how the medical device 200 controls and regulates the administration of a drug. Step 301 of the process shows the engagement of the activation unit 207 on the housing 225 of the medical device 200. After engagement of the activation unit 207, steps 303 and 305 occur either simultaneously or sequentially. Step 303 of the process shows the biometric reader 201 on the distal end of the housing 225 being activated and capturing a physical feature of the user. Step 305 of the process shows a sensor 229 in the housing of the medical device 200 detecting the angle at which the device 200 is held by the user. If the medical device 200 correctly verifies the user as the authorized user using the physical feature captured by the biometric reader 201 and verifies that the device 200 is held at an angle falling within a preselected range of acceptable angles, then step 307 of the process occurs. Step 307 of the process involves driving the drug dispensing unit 219 of the medical device 200 to a position that allows a therapeutic dose of the drug stored in the device 200 to pass through a mouthpiece 231 connected to the housing. Once the drug dispensing unit 219 has been located to a position that allows the drug to be administered, step 309 of the process occurs, this step involving the dispensing of the drug from the drug dispensing unit 219 through the mouthpiece 231 and directly into the authorized user's mouth.

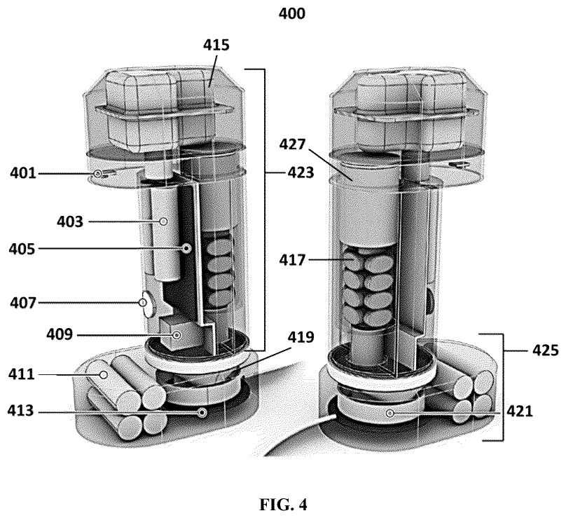

FIG. 4 depicts another exemplary embodiment of how the various components of a medical device 400 can be arranged. The housing 423 of the medical device 400 includes a biometric reader 401 at its distal end, the biometric reader 401 being configured to capture a facial image of the authorized user. The housing 423 further includes batteries 403 that are in communication with a driving unit 409 and a circuit board 405. The housing 423 also includes an activation unit 407 configured to initiate the biometric reader 401 to capture a physical feature of the user and initiate the medical device 400 to detect whether the drug stored in the drug storage compartment 417, located within the housing 423, is ready for administration. The housing also includes a storage unit 415 configured to store a life-saving drug product at its distal end and a locking mechanism 427 configured to prevent unauthorized access of the drug located in the drug storage compartment 417. The medical device 400 also includes a charging station 425 containing a sterilization unit 421 that is configured to interact with the drug dispensing unit 419 of the housing 423 and sterilize the drug dispensing unit 419. The charging dock also contains batteries 411 and a circuit board 413 configured to charge the batteries 403 located within the housing 423 when connected to an outside power source.

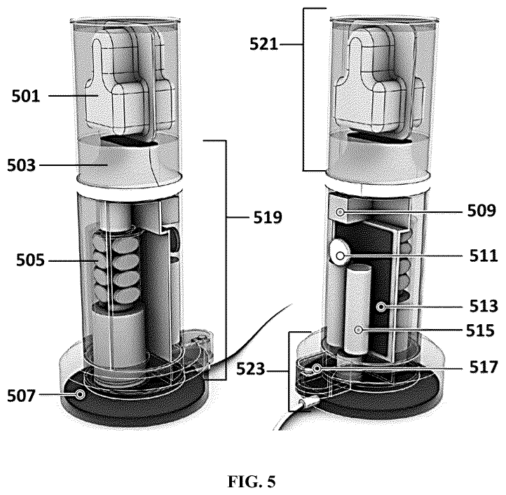

FIG. 5 depicts an additional exemplary embodiment of how the various components of a medical device 500 can be arranged. The medical device 500 includes a covering 521 that interacts with a drug dispensing unit 503. The covering 521 can be removed from the housing 519 of the medical device 500 to allow access to the drug dispensing unit 503. The covering 521 also includes a storage compartment 501 configured to store a life-saving drug product. The housing 519 of the medical device 500 includes batteries 515 that communicate with a driving unit 509 and a circuit board 513. The housing 519 also includes an activation unit 511 configured to initiate a biometric reader 517, located at the distal end of the housing 519, to capture a physical feature of the user and the medical device 500 to detect whether the drug stored in the drug storage compartment 505, located within the housing 519, is ready for administration. The medical device 500 also includes a charging dock 523 that is configured to interact with the distal end of the housing 519. The charging dock 523 contains a circuit board 507 configured to charge the batteries 515 located within the housing 519 when connected to an outside power source.

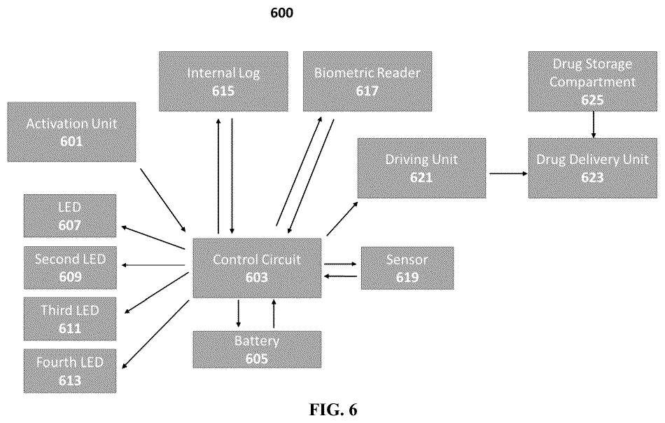

FIG. 6 depicts an operational overview of a medical device in accordance with an exemplary embodiment. The medical device 600 includes an activation unit 601. The activation unit 601 is in communication with a control circuit 603 residing within the medical device 600. The control circuit 603 is configured to communicate with at least one battery 605 in the medical device to supply power to the various components of the medical device 600. The control circuit is also configured to communicate with LED lights 607, 609, 611 and 613 to convey the status of the medical device 600 to the user accessing the medical device 600. The control circuit is also configured to communicate with an internal log 615 to at least determine when the medical device 600 was last used, whether the drug stored in the medical device 600 is ready for administration, whether the user accessing the medical device 600 is the authorized user and/or whether the device has been tampered with since its last use. The control circuit 603 is further configured to communicate with a biometric reader 617 to capture a physical feature of the user accessing the medical device 600. The control circuit 603 also communicates with at least one sensor 619 to determine the pressure within the medical device 600, the temperature within the medical device 600, and/or the angle at which the medical device 600 is being held. The control circuit 603 is also in communication with a driving unit 621 to control the administration of the drug in the medical device 600. The driving unit 621 communicates with a drug delivery unit 623 and translocates the drug delivery unit 623 to a position that allows the drug stored in a drug storage compartment 625 of the medical device 600 to be administered to the authorized user.

Any of the medical devices described herein can be used in decentralized clinical trials (e.g., does not involve visiting a clinic to use the device, can be operated at the authorized user's home) and/or be prescribed to the authorized user in a telehealth or telemedicine setting.

### Systems

The present disclosure also describes systems configured to control and monitor the administration of a drug to a prescribed user of the drug. The system can include (a) a medical device having a compartment configured to store one or more therapeutic doses of a drug, a drug delivery unit configured to dispense at least one therapeutic dose of the drug to an authorized user, at least one biometric reader attached to the medical device and configured to capture a physical feature of the authorized user, a sensor configured to measure an angle at which the medical device is being held, and a control circuit configured to (i) verify that the angle detected by the sensor is within a preset range of angles and, when the angle at which the medical device is being held is verified as falling with the preset range of angles, (ii) provide power to the at least one biometric reader and the drug delivery unit, wherein once the at least one biometric reader captures the physical feature of the authorized user and the control circuit verifies that the physical feature belongs to the authorized user, the control circuit instructs the drug delivery unit to dispense the therapeutic dose of the drug from the drug delivery unit to the authorized user; (b) a device configured to measure the authorized user's vitals and communicate with the medical device, and (c) a remote device configured to (i) wirelessly communicate with the control circuit of the medical device and (ii) execute an application program interface that allows the authorized user and a third-party to monitor a status of the medical device and adjust at least one of settings and control parameters of the medical device.

According to exemplary embodiments the medical device and the remote device can be configured to wirelessly communicate which may include a local area network (LAN), a wide area network (WAN), a wireless network (e.g., Wi-Fi), a mobile communication network, a satellite network, the Internet, optic fiber, coaxial cable, infrared, radio frequency (RF), Bluetooth, or any combination thereof. Other suitable communication and network types and configurations will be apparent to persons having skill in the relevant art.

In exemplary embodiments, the medical device can be programmed to log a video and/or photo of a user attempting to access the medical device and upload the video and/or photo to a mobile platform or wireless storage database.

In exemplary embodiments, the system includes a remote device configured to receive status information from the medical device and, from the status information, determine a number of therapeutic doses of a drug stored in at least one compartment of the medical device.

In exemplary embodiments, the system includes a remote device configured to receive status information from the medical device and, from the status information, determine whether the housing of medical device has been compromised or damaged.

In exemplary embodiments, the system includes a medical device equipped with a vitals detection unit configured to obtain vital measurements from an authorized user. The system can also include a remote device configured to wirelessly receive the vital measurements from the authorized user and determine, via an application program interface, whether the authorized user requires emergency care. The remote device can also be configured to automatically send notifications to emergency services or a licensed medical practitioner when the vital measurements indicate that the authorized user requires emergency care.

The device configured to measure the authorized user's vitals and communicate with the medical device can be either the vital detection unit of the medical device, a watch, a necklace, a wristband, an arm band or any other device known to be capable of detecting and transmitting information on a user's vitals. In exemplary embodiments, the device configured to measure the authorized user's vitals and communicate with the medical device can be programmed to perform an auditory and/or visual cue when the authorized user's vitals reach a critical state.

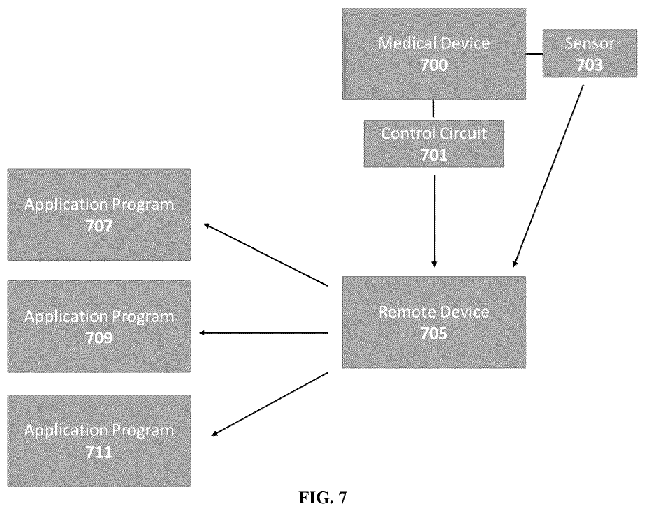

FIG. 7 depicts an additional exemplary embodiment of how the components of the systems can be arranged. A medical device 700 including a control circuit 701 and a sensor 703 is configured to wirelessly communicate with a remote device 705. The control circuit 701 transmits the status of the medical device 700 to the remote device 705. The status information transmitted to the remote device 705 can include the amount of drug remaining in the medical device 700, the identity of the user attempting to use the medical device 700, the vitals of the user attempting to use the medical device 700, the angle at which the medical device 700 is being held, the current pressure and temperature readings within the medical device 700, the battery life of the medical device 700, the amount of time remaining before the medical device 700 can administer another dose of the drug, the log history of the medical device 700 (e.g., the number of times the medical device 700 has been active in a set period of time, the number of authorized uses of the medical device 700 in a set period of time, the number of unauthorized attempts to use the medical device 700 in a set period of time, the number of times the drug compartment in the medical device 700 has been refilled). The sensor 703 can also directly wirelessly communicate with the remote device 705 and transmit the vitals of the user accessing the medical device 700 to the remote device 705.

The remote device 705 is configured to wirelessly transmit the status information from the medical device 700 and/or the vital information of the user accessing the medical device 700 to application programs 707, 709 and 711. These application programs can be installed on separate devices that are accessible to the authorized user, a licensed medical provider and/or an emergency services contact.

The remote device 705 can wirelessly transmit the information gathered by the medical device 700 to an application program 707 installed on a device accessible by the authorized user. The application program 707 can then at least inform the authorized user whether they can safely take the drug stored in the medical device 700 based on their vitals, when the drug stored in the medical device 700 is ready for administration, whether an attempt at unauthorized access of the drug stored in the medical device 700 has occurred and/or whether any tampering has been detected within the device.

The remote device 705 can also wirelessly transmit the information gathered by the medical device 700 to another application program 709 installed on a device accessible to a licensed medical practitioner. The application program 709 can then at least inform the licensed medical practitioner that the authorized user has safely taken the drug stored in the medical device 700, whether the medical device 700 has been tampered with, the vitals of the authorized user and/or whether an attempt at unauthorized access of the drug stored in the medical device 700 has occurred.

The remote device 705 can also wirelessly transmit the information gathered by the medical device 700 to another application program 711 installed on a device accessible to an emergency service provider. The application program 711 can then at least inform the emergency service provider that the authorized user is in critical condition and requires immediate emergency care.

### Methods

The present disclosure also describes methods for dispensing a drug through any of the programmable medical devices described herein. These methods can include: (i) detecting, by the sensor, an angle at which the medical device is being held by the authorized user, (ii) verifying, by the control circuit, that the angle detected by the sensor is within a preset range of acceptable angles, (iii) powering, by the control circuit, the at least one biometric and the drug delivery unit, (iv) verifying, by the control circuit, an identity of the authorized user based on a physical feature captured by the at least one biomarker reader, and (iv) instructing, by the control circuit, the drug delivery unit to dispense the therapeutic dose of the drug to the authorized user when both the identity of the authorized user is verified and the angle detected by the sensor is held is verified as falling with the preset range of acceptable angles.

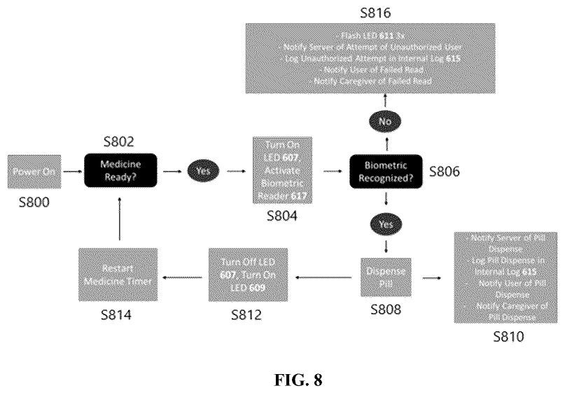

FIG. 8 depicts an exemplary method for how the medical device described in FIG. 6 can control the dispensing of a drug. The method includes powering the medical device 600 to allow the device to determine whether the drug contained in the drug storage compartment 625 is ready to be administered (S800). If the drug is ready to be administered (S802), a first visible LED 607 located on or within the medical device 600 will activate and display a first color to inform the user that the drug is ready to be administered (S804). The biometric reader 617 will then activate and begin capturing a physical feature of the user.

If the user is recognized as the authorized user (S806), then the medical device 600 will dispense the drug from the drug storage compartment 625 to the authorized user (S808). The medical device 600 can then notify an off-site server that the drug has been dispensed, log the time that the drug has been dispensed in the internal log 615, notify the authorized user that the drug has been dispensed and/or notify a caregiver or licensed medical practitioner that the drug has been dispensed (S810). After dispensing of the drug, the medical device 600 will inactivate the first visible LED 607 and activate a second visible LED 609 located on or within the medical device 600 to display a second color to indicate that the drug is not ready for administration (S812). The medical device 600 can then activate an internal clock or timer to determine when the drug can be administered to the authorized user again (S814).

If the user is not recognized as the authorized user, the medical device 600 can activate a third LED 611 located on or within the medical device 600 to display a third color to indicate that there was an attempt at unauthorized access of the drug stored in the drug storage compartment 625 (S816). The medical device 600 can then notify an off-site server, the authorized user, a caregiver and/or a licensed medical practitioner that an unauthorized attempt to access the drug has occurred. The medical device 600 can also log the time when the unauthorized attempt occurred in its internal log 615.

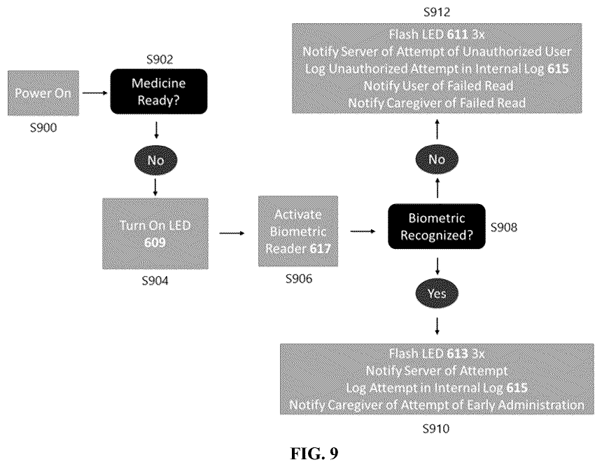

FIG. 9 depicts an exemplary method for how the medical device described in FIG. 6 can further control the dispensing of a drug. Once the medical device 600 is powered by the user (S900), the medical device 600 determines whether the drug stored in the drug storage compartment 625 is ready for administration (S902). If the drug is not ready for administration, the medical device 600 activates the second visible LED 609 located on or within the medical device 600 to display a second color to indicate that the drug is not ready for administration (S904). The medical device 600 then activates the biometric reader 615 (S906). If the biometric reader 615 identifies the user as an authorized user (S908), then the medical device 600 deactivates the second visible LED 609 and activates the third LED 611 to display the third color (S910). The medical device 600 then notifies an off-site server, a caregiver and/or a licensed medical practitioner that the authorized user attempted to access the drug in the drug storage compartment 625 before it was ready for administration.

If the user is not recognized as the authorized user (S908), the medical device 600 deactivates the second visible LED 609 and activates a fourth LED 613 located on or within the medical device 600 to display a fourth color to indicate that there was an attempt at unauthorized access of the drug stored in the drug storage compartment 625 (S912). The medical device 600 can then notify an off-site server, the authorized user, a caregiver and/or a licensed medical practitioner that an unauthorized attempt to access the drug has occurred. The medical device 600 can also log the time when the unauthorized attempt occurred in its internal log 615.

Any of the medical devices and/or systems disclosed herein can include computer program codes for performing the specialized functions described herein. The program code can be stored on a non-transitory computer usable medium, such as memory of the medical device or for the system (e.g., remote device, remote computing device, etc.), which may be memory semiconductors (e.g., DRAMs, etc.) or other tangible non-transitory means for providing software to the system. The computer programs may also be received via a communications interface. Such computer programs, when executed, may enable the systems to implement the present methods and exemplary embodiments discussed herein. Accordingly, such computer programs may represent controllers of the system. Where the present disclosure is implemented using software, the software may be stored in a computer program product or non-transitory computer readable medium and loaded into the system using a removable storage drive, an interface, a hard disk drive, or communications interface, where applicable.

The processing device of any one of the medical devices disclosed herein can include one or more modules or engines configured to perform the functions of the exemplary embodiments described herein. Each of the modules or engines may be implemented using hardware and, in some instances, may also utilize software, such as corresponding to program code and/or programs stored in memory. In such instances, program code may be interpreted or compiled by the respective processors (e.g., by a compiling module or engine) prior to execution. For example, the program code may be source code written in a programming language that is translated into a lower-level language, such as assembly language or machine code, for execution by the one or more processors and/or any additional hardware components. The process of compiling may include the use of lexical analysis, preprocessing, parsing, semantic analysis, syntax-directed translation, code generation, code optimization, and any other techniques that may be suitable for translation of program code into a lower-level language suitable for controlling any system disclosed herein to perform the any of the functions disclosed herein. It will be apparent to persons having skill in the relevant art that such processes result in the systems including specially configured computing devices uniquely programmed to perform the functions of any embodiment described herein.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

It will be appreciated by those skilled in the art that features from the various exemplary embodiments can be combined to create other embodiments of the medical devices, methods and systems described herein.

What is claimed is:

1. A programmable medical device comprising:
(a) a housing that includes at least one compartment configured to store a drug,
(b) a drug delivery unit mounted within the housing that is configured to store a therapeutic dose of the drug from the at least one compartment and dispense the therapeutic dose to an authorized user,
(c) at least one biometric reader configured to capture a physical feature of the authorized user,
(d) a sensor configured to detect an angle at which the medical device is being held by the authorized user, and
(e) a control circuit configured to (i) communicate with the sensor and verify that the angle detected by the sensor is within a preset range of angles and, when the angle is verified as falling within the preset range of angles, (ii) provide power to the at least one biometric reader and the drug delivery unit, wherein once the at least one biometric reader captures the physical feature of the authorized user and the control circuit verifies that the physical feature belongs to the authorized user, the control circuit instructs the drug delivery unit to dispense the therapeutic dose of the drug from the drug delivery unit to the authorized user.

2. The medical device of claim 1, comprising:
a circuit board configured to control and monitor an operation of the medical device, wherein the control circuit is mounted to the circuit board.

3. The medical device of claim 1, comprising:
a driving unit within the housing, the driving unit configured to drive one or more components of the drug delivery unit to administer the drug to the authorized user; and
a battery mounted within the housing and configured to supply the circuit board and the driving unit with operating power.

4. The medical device of claim 3, comprising:
a charging dock configured to mechanically engage with the housing and charge the battery mounted within the housing.

5. The medical device of claim 3, comprising:
a charging port on the housing configured to communicate with an external power source and charge the battery mounted within the housing.

6. The medical device of claim 1, wherein the device includes a locking device disposed on a face of the housing and configured to prevent the authorized user and an unauthorized user from accessing the drug in the at least one compartment.

7. The medical device of claim 3, wherein the control circuit is configured to actuate the driving unit to drive the one or more components of the drug delivery unit to administer the therapeutic dose of the drug to the authorized user when the verification is successful and when a pre-defined time interval is met.

8. The medical device of claim 1, comprising a life-saving drug product disposed in a storage unit located in the housing.

9. The medical device of claim 8, wherein the life-saving drug product includes opioid antagonists.

10. The medical device of claim 8, wherein the storage unit is housed in the at least one compartment.

11. The medical device of claim 8, wherein the housing includes a plurality of compartments and at least one of the compartments is the storage unit, the storage unit being separate from and adjacent (above, below, beside) to the at least one compartment storing the drug.

12. The medical device of claim 1, comprising:
at least one transceiver configured for wireless communication with a global positioning system,
wherein the control circuit is configured to determine a location of the medical device including at least one of a life-saving drug product and an antidote based on geolocation data received from the global positioning system (GPS).

13. The medical device of claim 3, wherein the at least one compartment includes:
a tray having plural chambers, wherein each chamber is configured to store the therapeutic dose of the drug in oral dosage form, wherein the tray is configured to rotate when driven by the driving unit, and
an opening to allow the therapeutic dose to be dispensed when one of the plural chambers is aligned with the opening.

14. The medical device of claim 6, wherein the locking device is integrated into the housing and is configured to allow access to the at least one compartment when interfacing with an authorized key.

15. The medical device of claim 3, wherein the at least one compartment is a tablet hopper configured to store plural units of the drug in oral dosage form, wherein one oral dosage form of the drug contains the therapeutic dose, and wherein the one or more components of the drug delivery unit includes:
a door that is configured to be driven by the driving unit to allow one or more oral dosage forms of the drug to be dispensed from the at least one compartment when the driving unit is actuated by the control circuit.

16. The medical device of claim 15, wherein the authorized key is an electronic key.

17. The medical device of claim 15, wherein the authorized key is a physical key.

18. The medical device of claim 1, wherein the drug delivery unit includes a mouthpiece configured to fit in the user's mouth and dispense the therapeutic dose of the drug directly into the authorized user's mouth.

19. The medical device of claim 1, wherein the at least one biometric reader is configured to capture the authorized user's fingerprint, and wherein the control circuit is configured to verify an identity of the authorized user by comparing the captured fingerprint to a stored fingerprint of the authorized user.

20. The medical device of claim 1, wherein the at least one biometric reader is configured to capture an ocular biometry of the authorized user, and wherein the control circuit is configured to verify an identity of the authorized user by comparing the captured ocular biometry to a stored ocular biometry of the authorized user.

21. The medical device of claim 1, wherein the at least one biometric reader is configured to capture a lip pattern of the authorized user, and wherein the control circuit is configured to verify an identity of the authorized user by comparing the captured lip pattern to a stored lip pattern of the authorized user.

22. The medical device of claim 1, comprising a dosage splitting unit configured to split the therapeutic dose of the drug stored in the at least one compartment into smaller dosages.

23. The medical device of claim 22, wherein the dosage splitting unit includes a blade.

24. The medical device of claim 1, wherein the housing is configured for portability by the authorized user.

25. The medical device of claim 1, comprising a transceiver configured for communication with a wireless network.

26. The medical device of claim 25, wherein the transceiver is configured to wirelessly communicate with a remote device associated with the authorized user.

27. The medical device of claim 26, wherein the remote device includes a second sensor that obtains vital measurements from the authorized user.

28. The medical device of claim 26, wherein the remote device of the authorized user is configured to send the vital measurements of the authorized user to a third-party remote device.

29. The medical device of claim 1, comprising:
a sterilization unit configured to sterilize the drug delivery unit or a mouthpiece before and/or after dispensing the therapeutic dose of the drug.

30. The medical device of claim 18, wherein the medical device comprises two biometric readers, one biometric reader being located at or near the distal end of the housing and configured to capture a facial feature of the authorized user and the other biometric reader being located on the mouthpiece and configured to capture a lip pattern of the authorized user.

31. The medical device of claim 30, wherein the control circuit is configured to (i) communicate with the sensor and verify that the angle detected by the sensor is within a preset range of angles and, when the angle is verified as falling within the preset range of angles, (ii) provide power to the two biometric readers and the drug delivery unit, wherein once the two biometric readers capture the lip pattern and the facial feature of the authorized user and the control circuit verifies that the lip pattern and the facial feature belong to the authorized user, the control circuit instructs the drug delivery unit to dispense the therapeutic dose of the drug from the drug delivery unit into the mouth of the authorized user.

32. The medical device of claim 1, wherein the medical device is configured to improve drug administration to authorized users with limited motor skills or dysphagia.

33. A system comprising:
(a) a medical device having a compartment configured to store one or more therapeutic doses of a drug, a drug delivery unit configured to dispense at least one therapeutic dose of the drug to an authorized user, at least one biometric reader configured to capture a physical feature of the authorized user, a sensor configured to measure an angle at which the medical device is being held, and a control circuit configured to (i) verify that the angle detected by the sensor is within a preset range of angles and, when the angle at which the medical device is being held is verified as falling with the preset range of angles, (ii) provide power to the at least one biometric reader and the drug delivery unit, wherein once the at least one biometric reader captures the physical feature of the authorized user and the control circuit verifies that the physical feature belongs to the authorized user, the control circuit instructs the drug delivery unit to dispense the therapeutic dose of the drug from the drug delivery unit to the authorized user;
(b) a device configured to measure the authorized user's vitals and communicate with the medical device, and
(c) a remote device configured to (i) wirelessly communicate with the control circuit of the medical device and (ii) execute an application program interface that allows the authorized user and a third-party to monitor a status of the medical device and adjust at least one of settings and control parameters of the medical device.

34. The system of claim 33, wherein the remote device is configured to:
receive status information from the medical device; and
from the status information:
determine a number of therapeutic doses remaining to be dispensed;
determine whether the housing has been compromised or damaged.

35. The system of claim 34, comprising:
a sensor configured to obtain vital measurements from the authorized user.

36. The system of claim 35, wherein the remote device is configured to wirelessly receive the vital measurements from the authorized user and determine, via the application program interface, whether the authorized user requires emergency care.

37. The system of claim 36, wherein the remote device is configured to send automatically send notification to emergency services or a physician when the vital measurements indicate that the authorized user requires emergency care.

38. The medical device of claim 1, comprising:
at least one transceiver located within the storage unit and configured for wireless communication with a global positioning system,
wherein the control circuit is configured to determine a location of the storage unit based on geolocation data received from the global positioning system (GPS).

39. The medical device of claim 1, wherein the at least one biometric reader is configured to capture a facial image of the authorized user, and wherein the control circuit is configured to verify an identity of the authorized user by comparing features of the captured facial image to features of a stored facial image of the authorized user.

40. The medical device of claim 26, wherein the transceiver is configured to wirelessly communicate with a secondary remote device associated with a secondary user.

41. The medical device of claim 1, wherein the device comprises at least one tamper detection sensor that is configured to detect at least an attempt at unauthorized access to the at least one compartment of the drug delivery unit.

42. The medical device of claim 41, wherein the at least one compartment is a pressurized chamber, and the at least one tamper detection sensor is a pressure sensor configured to monitor the pressure of the at least one compartment, and wherein the control circuit is configured to receive pressure readings from the pressure sensor and compare the received pressure reading to a threshold to determine whether the at least one compartment has been tampered.

43. The medical device of claim 42, wherein the at least one compartment comprises an inner shell and an outer shell of the housing, wherein inner shell is the pressurized chamber.

44. The medical device of claim 1, wherein the medical device comprises at least one storage condition monitoring sensor configured to detect environmental conditions in the at least one compartment configured to store the drug.

45. The medical device of claim 44, wherein the at least one storage condition monitoring sensor is either a temperature sensor configured to detect the temperature in the at least one compartment configured to store the drug or is a humidity sensor configured to detect the humidity or moisture levels in the at least one compartment configured to store the drug.

46. The medical device of claim 1, wherein the device comprises a destruction unit located within the housing, the destruction unit being configured to mechanically and/or chemically destroy an amount of drug remaining in the at least one compartment once a tampering attempt has occurred.

47. A method for dispensing a drug through a programmable medical device that includes that includes (a) a housing having at least one compartment configured to store a drug, (b) a drug delivery unit mounted within the housing, (c) at least one biometric reader, (d) a sensor configured to measure an angle at which the medical device is being held and (e) a control circuit connected to the drug delivery unit, the at least one biometric reader, and the sensor, the method comprising:
(i) detecting, by the sensor, an angle at which the medical device is being held by the authorized user,
(ii) verifying, by the control circuit, that the angle detected by the sensor is within a preset range of acceptable angles,
(iii) powering, by the control circuit, the at least one biometric and the drug delivery unit,
(iv) verifying, by the control circuit, an identity of the authorized user based on a physical feature captured by the at least one biomarker reader, and
(iv) instructing, by the control circuit, the drug delivery unit to dispense the therapeutic dose of the drug to the authorized user when both the identity of the authorized user is verified and the angle detected by the sensor is held is verified as falling with the preset range of acceptable angles.
